(19) United States
(12) Patent Application Publication
(10) Pub. No.: US 2016/0069871 A1
(43) Pub. Date: Mar. 10, 2016

(54) MAGNETIC BEAD, LIGAND-BINDING BEAD, METHOD FOR DETECTING OR SEPARATING TARGET SUBSTANCE, AND METHOD FOR PRODUCING THE MAGNETIC BEAD

(71) Applicants: JSR CORPORATION, Minato-ku (JP); JSR Life Sciences Corporation, Minato-ku (JP)

(72) Inventor: Yuuichi UEYA, Minato-ku (JP)

(73) Assignees: JSR CORPORATION, Minato-ku (JP); JSR Life Sciences Corporation, Minato-ku (JP)

(21) Appl. No.: 14/847,100

(22) Filed: Sep. 8, 2015

(30) Foreign Application Priority Data
Sep. 8, 2014 (JP) 2014-182071

(51) Int. Cl. G01N 33/543; C07K 17/08; C08F 285/00

(52) U.S. Cl. CPC G01N 33/5434; C08F 285/00; C07K 17/08

(57) ABSTRACT

A magnetic bead, formed by binding a chain polymer at least to the surface, in which the chain polymer is a chain polymer which has a hydrophilic repeating unit, and has a group comprising a reactive functional group at the end of the side, to which the magnetic bead does not bind, through an imino group or an N-substituted imino group, and a density of the chain polymer occupying the surface of the magnetic bead is 0.1 polymers/nm² or more.

### FIELD OF THE INVENTION

The present invention relates to a magnetic bead, a ligand-binding bead, a method for detecting or separating a target substance, and a method for producing the magnetic bead.

### BACKGROUND OF THE INVENTION

A solid-phase support such as magnetic bead and a sensor chip is used for the purpose of detecting and separating a target substance such as protein, nucleic acid and cell from sample such as blood. As the method of detection and separation using a solid-phase support, a general method is that a ligand is allowed to bind to a solid-phase support, with which a sample is brought into contact to allow a target substance to react with the ligand. At the time of the above contact, however, the target substance and impurities in the sample nonspecifically adsorb on the surface of the solid-phase support instead of the ligand, which can cause noise to decrease sensitivity in detection.

Accordingly, it is known that a magnetic bead in which a poly(hydroxyethyl methacrylamide)-poly(methacrylic acid) block copolymer, for example, is introduced on the surface by atom transfer radical polymerization (ATRP) to form a polymer brush (JP 2009-542862 W).

In the above magnetic bead, however, a number of reactive functional groups such as carboxy group continuously exist on a side chain of each copolymer bound on the surface of magnetic bead, and therefore a ligand may bind to the reactive functional group at multiple points. When a ligand binds at multiple points as described above, the original structure of the ligand is broken and there may be a possibility for lowering its activity. When a reactive functional group is introduced by ATRP, an amount of the reactive functional group easily increases and decreases depending on slight changes in reaction conditions (such as reaction time, reaction temperature and oxygen concentration in the system), and it is not easy to obtain the desired bead with good reproducibility.

In the meantime, a gold evaporation substrate in which a chain polymer having a carboxyalkylthio group introduced in the end binds to the surface (JP 4818056 B1) and a gold evaporation substrate in which a chain polymer having a hydroxyethylamino group introduced in the end binds to the surface (Biomacromolecules 2013, 14, 3294-3303) are known as a structure in which a chain polymer having a reactive functional group at the end binds to the surface.

### SUMMARY OF THE INVENTION

Under the above circumstances, the present inventors introduced a carboxyalkylthio group in the end of a chain polymer binding to the surface of magnetic beads, and consequently found that degeneration accompanied with color changes is brought about.

It is considered that a magnetic substance contained in magnetic beads is reduced by a thiol group-containing compound used for the above introduction to cause color changes, and there may be a possibility for a change in magnetic performance.

Therefore, an object of the present invention is to provide a magnetic bead in which nonspecific adsorption is hard to occur, degeneration accompanied by color changes is not brought about, and a chain polymer having a reactive functional group at the end binds to the surface.

As a result of intensive investigation, the present inventors found that a magnetic bead, in which not only nonspecific adsorption is hard to occur but also degeneration accompanied by color changes is not brought about, is obtained by introducing a reactive functional group in the end of the magnetic bead, formed by binding a chain polymer having a hydrophilic repeating unit to the surface at a specific density, through a specific linking group such as an imino group or an N-substituted imino group, thereby completing the present invention.

That is, the present invention provides <1> a magnetic bead, formed by binding a chain polymer at least to the surface, in which the chain polymer is a chain polymer which has a hydrophilic repeating unit, and has a group comprising a reactive functional group at the end of the side, to which the magnetic bead does not bind, through an imino group or an N-substituted imino group, and a density of the chain polymer occupying the surface of the magnetic bead is 0.1 polymers/n or more.

The present invention also provides <2> a ligand-binding bead, formed by binding a ligand to the magnetic bead according to <1> above.

The present invention further provides <3> a method for detecting or separating a target substance in a sample, the method being characterized by using the ligand-binding bead according to <2> above.

The present invention further provides <4> a method for producing the magnetic bead according to <1> above, the method being characterized by comprising (Step 1) a step of preparing a magnetic bead material having a polymerization initiating group at least on the surface, (Step 2) a step of polymerizing a hydrophilic monomer from the polymerization initiating group, and (Step 3) a step of allowing a compound having a primary or secondary amino group to react with the end of a chain polymer formed on the surface of the magnetic bead material in the step 2.

### Effect of the Invention

In the magnetic bead of the present invention, a target substance and impurities in a sample are hard to adsorb on the surface, and nonspecific adsorption is suppressed, and degeneration accompanied by color changes is not brought about. Further, when a target substance is detected and separated from a sample, a sufficient amount of ligand can be allowed to bind to the magnetic bead.

According to the production method of the present invention, the magnetic bead, in which nonspecific adsorption is hard to occur, and degeneration accompanied by color changes is not brought about, to which, when a target substance is detected and separated from a sample, a sufficient amount of ligand can be allowed to bind, can be simply produced.

### DETAILED DESCRIPTION OF THE INVENTION

### <Magnetic Bead>

The magnetic beads of the present invention, formed by binding a chain polymer at least to the surface, are characterized in that the chain polymer is a chain polymer which has a hydrophilic repeating unit, and has a group comprising a reactive functional group at the end of the side, to which the magnetic beads do not bind, through an imino group (—NH—) or an N-substituted imino group, and a density of the chain polymer occupying the surface of the magnetic beads is 0.1 polymers/nm² or more. At the outset, the magnetic beads of the present invention is described in detail.

(Hydrophilic Repeating Unit)

As the hydrophilic repeating unit, a repeating unit having a hydrophilic group is preferred, and a repeating unit having a hydrophilic group on a side chain is more preferred.

Examples thereof include a repeating unit derived from a (meth)acrylate monomer having a hydrophilic group, a repeating unit derived from a (meth)acrylamide monomer having a hydrophilic group, and a repeating unit derived from a styrene monomer having a hydrophilic group. Among these repeating units, a repeating unit derived from a (meth)acrylate monomer having a hydrophilic group, and a repeating unit derived from a (meth)acrylamide monomer having a hydrophilic group are preferred in terms of the suppression of nonspecific adsorption.

Examples of the hydrophilic group include a hydroxy group, an alkoxy group, a polyoxyalkylene group, a group having a zwitterionic structure, a sulfonyl group, a sulfinyl group and a phosphate group, and a structural unit may have one of these groups or may have two or more of these groups. As the alkoxy group, an alkoxy group having one or two carbons is preferred. Examples thereof include, for example, a methoxy group and an ethoxy group.

Among these groups, as the hydrophilic group, a hydroxy group, a group having a zwitterionic structure, a polyoxyalkylene group and a phosphate group are preferred, and a hydroxy group, a group having a zwitterionic structure and a polyoxyalkylene group are more preferred in terms of suppressing nonspecific adsorption.

As the above polyoxyalkylene group, a group represented by —(R^{a}O)_{q}—(R^{a} represents an alkanediyl group and q represents an integer from 2 to 100, and q groups of R^{a}s may be the same or different) is preferred.

The number of carbons in an alkanediyl group represented by R^{a} is preferably from 2 to 4, more preferably 2 or 3 and especially preferably 2.

In addition, the alkanediyl group represented by R^{a} may be a straight chain or a branched chain, and specific examples thereof include, for example, an ethane-1,2-diyl group, a propane-1,2-diyl group, a propane-1,3-diyl group and a propane-2,2-diyl group. Among these groups, an ethane-1,2-diyl group is preferred.

q represents an integer from 2 to 100, and an integer from 3 to 80 is preferred, an integer from 4 to 60 is more preferred, an integer from 5 to 40 is further preferred, an integer from 6 to 30 is further preferred, and an integer from 7 to 20 is especially preferred.

As the above group having a zwitterionic structure, an organic group having a quaternary ammonium salt cationic functional group and a monovalent or divalent anionic functional group selected from the group consisting of —(C═O)O⁻, —SO₃⁻ and —O— (O═P—O⁻)—O— is preferred, an organic group represented by the following formula (1) or (2) is more preferred, and an organic group represented by the following formula (1) is especially preferred in terms of suppressing nonspecific adsorption.

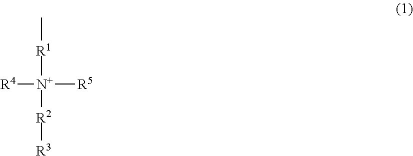

[In the formula (1),

R¹ and R² each independently represent a single bond or a divalent organic group having 1 to 10 carbons,

R³ represents —(C═O)O⁻ or —SO₃⁻, and

R⁴ and R⁵ each independently represent a methyl group or an ethyl group.]

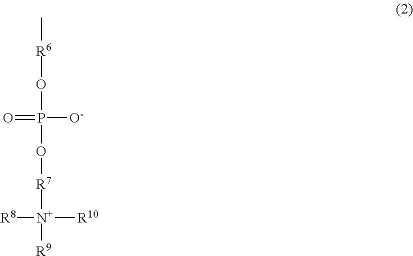

[In the formula (2),

R⁶ and R⁷ each independently represent a single bond or a divalent organic group having 1 to 10 carbons, and

R⁸, R⁹ and R¹⁰ each independently represent a methyl group or an ethyl group.]

R¹ and R² in the formula (1) and R⁶ and R⁷ in the formula (2) each independently represent a single bond or a divalent organic group having from 1 to 10 carbons, and in terms of suppressing nonspecific adsorption, a divalent organic group having from 1 to 10 carbons is preferred, a divalent hydrocarbon group having from 1 to 10 carbons and a group having one or more bonds selected from the group consisting of an ether bond, an amide bond and an ester bond between carbon and carbon atoms in a divalent hydrocarbon group having from 2 to 10 carbons is more preferred, and a divalent hydrocarbon group having from 1 to 10 carbons is especially preferred.

When the divalent organic group is a divalent hydrocarbon group, the number of carbons is preferably from 1 to 8, more preferably from 1 to 6, further preferably from 1 to 4, and especially preferably from 1 to 3. Meanwhile, when the divalent organic group is a group having one or more bonds selected from the group consisting of an ether bond, an amide bond and an ester bond between carbon and carbon atoms in a divalent hydrocarbon group, the number of carbons of the divalent hydrocarbon group in such a group is preferably from 2 to 8, more preferably from 2 to 6, further preferably from 2 to 4, and especially preferably 2 or 3.

As the “divalent hydrocarbon group” in R¹, R², R⁶ and R⁷, a divalent aliphatic hydrocarbon group is preferred. Such a divalent aliphatic hydrocarbon group may be a straight chain or a branched chain.

As the above divalent aliphatic hydrocarbon group, an alkanediyl group is preferred and specific examples thereof include, for example, a methane-1,1-diyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,2-diyl group, a butane-1,3-diyl group, a butane-1,4-diyl group, a pentane-1,4-diyl group, a pentane-1,5-diyl group, a hexane-1,5-diyl group and a hexane-1,6-diyl group.

R³ in the formula (1) is preferably —(C═O)O⁻.

R⁴ and R⁵ in the formula (1) and R⁸, R⁹ and R¹⁰ in the formula (2) are preferably a methyl group.

In addition, suitable specific examples of the hydrophilic repeating unit include a repeating unit represented by the following formula (3).

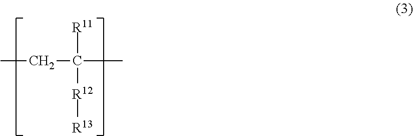

[In the formula (3),

R¹¹ represents a hydrogen atom or a methyl group,

R¹² represents —(C═O)—O—*, —(C═O)—NR¹⁴—*, where R¹⁴ represents a hydrogen atom or a methyl group, and * represents a position bound to R¹³ in the formula (3), or a phenylene group, and

R¹³ represents a group having a zwitterionic structure, an organic group having a hydroxy group, or an organic group having a polyoxyalkylene group.]

In the formula (3), R¹² is preferably —(C═O)—O—* or —(C═O)—NH—* in terms of suppressing nonspecific adsorption.

The group having a zwitterionic structure represented by R¹³ is the same as the above groups having a zwitterionic structure.

Examples of the organic group having a hydroxy group represented by R¹³ include a group represented by the following formula (4).

—R¹⁵—OH (4)

[In the formula (4), R¹⁵ represents a divalent organic group.]

Examples of the divalent organic group represented by R¹⁰ include a divalent hydrocarbon group and a group having one or more bonds selected from the group consisting of an ether bond, an amide bond and an ester bond between carbon and carbon atoms in a divalent hydrocarbon group having 2 or more carbons, and a divalent hydrocarbon group is preferred.

When the divalent organic group is a divalent hydrocarbon group, the number of carbons is preferably from 1 to 8, more preferably from 1 to 6, further preferably from 1 to 4, and especially preferably from 1 to 3. Meanwhile, when the divalent organic group is a group having one or more selected from the group consisting of an ether bond, an amide bond and an ester bond between carbon and carbon atoms in a divalent hydrocarbon group having 2 or more carbons, the number of carbons of the divalent hydrocarbon group in such a group is preferably from 2 to 8, more preferably from 2 to 6, further preferably from 2 to 4, and especially preferably 2 or 3.

As the “divalent hydrocarbon group” in R¹⁵, a divalent aliphatic hydrocarbon group is preferred. Such a divalent aliphatic hydrocarbon group may be a straight chain or a branched chain.

As the above divalent aliphatic hydrocarbon group, an alkanediyl group is preferred and specific examples thereof include, for example, a methane-1,1-diyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,2-diyl group, a butane-1,3-diyl group, a butane-1,4-diyl group, a pentane-1,4-diyl group, a pentane-1,5-diyl group, a hexane-1,5-diyl group, and a hexane-1,6-diyl group.

As the organic group having a polyoxyalkylene group represented by R¹³, a group represented by —(R^{a}O)_{q}—R^{b} is preferred, where R^{b} represents an alkyl group having 1 to 4 carbons. R^{a} and q have the same meaning as above, R^{d} represents an alkanediyl group and q represents an integer from 2 to 100, respectively.

The number of carbons in an alkyl group represented by R^{b} is preferably 1 to 3 and more preferably 1 or 2. In addition, the alkyl group represented by R^{b} may be a straight chain or a branched chain, and specific examples thereof include a methyl group, an ethyl group, a n-propyl group, an isopropyl group, a n-butyl group, an isobutyl group, a sec-butyl group and a tert-butyl group, and a methyl group is especially preferred.

In addition, the chain polymer may have a repeating unit other than the above hydrophilic repeating unit, and a hydrophilic polymer is preferred, and a homopolymer of the above hydrophilic repeating unit is more preferred. In addition, a chain vinyl polymer is preferred.

In the present description, hydrophilicity means to have a strong affinity for water. Specifically, when a homopolymer comprising only one repeating unit (the number average molecular weight by a method of measurement in examples is about from 1,000 to 100,000) is dissolved in an amount of 1 g or more in 100 g of pure water at normal temperature (25° C.), the repeating unit is hydrophilic.

(End Structure)

The above chain polymer has a group comprising a reactive functional group at the end of the side, to which the magnetic beads do not bind, through an imino group (—NH—) or an N-substituted imino group. When such an end structure is shown with an imino group or an N-substituted imino group, the structure is as the following formula (5).

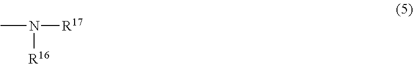

[In the formula (5),
R¹⁶ represents a hydrogen atom or a substituent, and

R¹⁷ represents a group comprising a reactive functional group.]

Examples of the substituent represented by R¹⁶ include an alkyl group. The number of carbons in such alkyl group is preferably from 1 to 4 and more preferably 1 or 2. The alkyl group may be a straight chain or a branched chain, and specific examples thereof include, for example, a methyl group, an ethyl group, a n-propyl group, an isopropyl group, a n-butyl group, an isobutyl group, a sec-butyl group and a tert-butyl group.

R¹⁶ is preferably a hydrogen atom in terms of efficient binding of a ligand.

R¹⁷ represents a group comprising a reactive functional group. Examples of the reactive functional group include a carboxy group, a tosyl group, an amino group, an epoxy group, an acyl group and an azide group, and one or two or more of these groups may be contained. Among these groups, a carboxy group, a tosyl group, a primary amino group and an epoxy group are preferred in terms of preventing a bound ligand from coming off and from the point that, when a biomolecule such as a protein or a nucleic acid is used as a ligand, the biomolecule can bind to magnetic beads using a functional group which the ligand originally has, and a carboxy group is more preferred, for example, from the point that a ligand is easily allowed to simply and quickly bind to magnetic beads.

As the group comprising a reactive functional group represented by R¹⁷, an organic group represented by the following formula (6) is preferred.

—R¹⁸—Y (6)

[In the formula (6),

R¹⁸ represents a divalent organic group, and

Y represents a reactive functional group.]

Examples of the divalent organic group represented by R¹⁸ include a divalent hydrocarbon group and a group having one or more selected from the group consisting of an ether bond, an amide bond and an ester bond between carbon and carbon atoms in a divalent hydrocarbon group having 2 or more carbons.

When the divalent organic group is a divalent hydrocarbon group, the number of carbons is preferably from 1 to 10, more preferably from 1 to 8, and especially preferably from 1 to 6. Meanwhile, when the divalent organic group is a group having one or more selected from the group consisting of an ether bond, an amide bond and an ester bond between carbon and carbon atoms in a divalent hydrocarbon group having two or more carbons, the number of carbons of the divalent hydrocarbon group in such a group is preferably from 2 to 10, more preferably from 2 to 8, and especially preferably from 2 to 6.

As the “divalent hydrocarbon group” in R¹⁸, a divalent aliphatic hydrocarbon group is preferred. Such a divalent aliphatic hydrocarbon group may be a straight chain or a branched chain.

As the above divalent aliphatic hydrocarbon group, an alkanediyl group is preferred and specific examples thereof include, for example, a methane-1,1-diyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,2-diyl group, a butane-1,3-diyl group, a butane-1,4-diyl group, a pentane-1,4-diyl group, a pentane-1,5-diyl group, a hexane-1,5-diyl group, a hexane-1,6-diyl group.

As the group having one or more selected from the group consisting of an ether bond, an amide bond and an ester bond between carbon and carbon atoms in a divalent hydrocarbon group having two or more carbons, a group having an ester bond between carbon and carbon atoms in the divalent hydrocarbon group having 2 or more carbons is preferred, and a divalent group represented by —R^{c}—O(C═O)—R^{d}—*, where R^{c} and R^{d} each independently represent an alkanediyl group having 2 to 4 carbons, and * represents a position bound to Y in the formula (6), is more preferred, for example, in terms of obtaining a chain polymer in a simple manner. The number of carbons in an alkanediyl group is preferably 2 or 3 and more preferably 2. The alkanediyl group may be a straight chain or a branched chain, and examples thereof include an ethane-1,2-diyl group, a propane-1,2-diyl group and a propane-1,3-diyl group.

In addition, a content of the reactive functional group is preferably 0.7 μmol or more, more preferably 1 μmol or more, further preferably 2 μmol or more, and also preferably 50 μmol or less, more preferably 40 μmol or less, and further preferably 30 μmol or less per g of solid content in magnetic beads in terms of the amount of bound ligand.

The content of the reactive functional group can be, for example when the reactive functional group is a carboxy group, measured by, for example, conductometry, and specifically can be measured in accordance with a method described in the Examples mentioned below. In addition, when the reactive functional group is a tosyl group, the content can be obtained by, for example, measuring the ultraviolet-visible light absorption of the tosyl group introduced in magnetic beads, and when the reactive functional group is an amino group, the content can be obtained by, for example, allowing an amino group to react with N-succinimidyl-3-(2-pyridyldithio) propionate, followed by reduction, and measuring the absorbance of free thiopyridyl groups.

The end opposite to the above side of a chain polymer is not particularly limited as long as the end binds to the surface of magnetic beads, and it is preferred that the end bind to the surface of magnetic beads through a divalent linking group comprising a residual group of a polymerization initiating group. As the polymerization initiating group, a polymerization initiating group capable of living polymerization is preferred, a living radical polymerization initiating group is more preferred, an atom transfer radical polymerization initiating group and a reversible addition-fragmentation chain transfer polymerization initiating group are further preferred, and an atom transfer radical polymerization initiating group is especially preferred. Examples of the divalent linking group comprising a residual group of an atom transfer radical polymerization initiating group include a divalent group represented by the following formula (7-1) or (7-2).

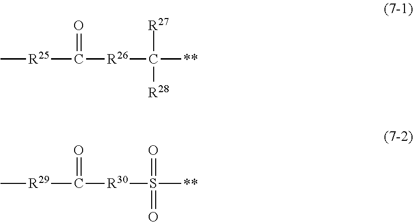

[In the above formula,

R¹⁹ and R²³ represent —O— or —NH—,

R²⁰ and R²⁴ each independently represent a single bond or a phenylene group,

R²¹ and R²² each independently represent a hydrogen atom or an alkyl group, and

** represents a position bound to the end of a chain polymer.]

R¹⁹ and R²³ are preferably —O—, R²⁰ and R²⁴ are preferably a single bond, and R²⁴ and R²² are preferably an alkyl group.

The number of carbons in the alkyl group represented by R²¹ and R²² is preferably from 1 to 8, more preferably from 1 to 4, and especially preferably 1 or 2. The alkyl group may be a straight chain or a branched chain, and specific examples thereof include, for example, a methyl group, an ethyl group, a n-propyl group, an isopropyl group, a n-butyl group, an isobutyl group, a sec-butyl group, a tert-butyl group, a pentyl group, a hexyl group, a heptyl group and an octyl group.

In the magnetic beads of the present invention, a density of the above chain polymer occupying the surface of magnetic beads is 0.1 polymers/nm² or more. That is, a polymer brush constituted from chain polymers is formed on the surface of magnetic beads, and by the above constitution, the beads can bind to a sufficient amount of ligand and the effect of suppressing nonspecific adsorption is improved.

The density of the above chain polymer is preferably 0.3 polymers/nm² or more, more preferably 0.4 polymers/nm² or more, and further preferably 0.6 polymers/nm² or more in terms of suppressing nonspecific adsorption and in terms of the amount of bound ligand, and also preferably 2 polymers/nm² or less, more preferably 1.6 polymers/nm² or less, and further preferably 1.2 polymers/nm² or less in terms of forming a polymer brush in an ease manner.

The density of the above chain polymer can be calculated, for example, by the following formula. Specifically, chain polymers are released from magnetic beads by, for example, hydrolysis, and the density can be measured in accordance with a method described in the examples mentioned below.

Density of chain polymer (chain polymers/nm²)=Number of chain polymers binding to 1 g of beads (chain polymers)/Total surface area of 1 g of beads (nm²)

The number average molecular weight (Mn) of chain polymer is preferably from 1,000 to 100,000, more preferably from 3,000 to 50,000, and especially preferably from 5,000 to 30,000.

The weight average molecular weight (Mw) of chain polymer is preferably from 1,000 to 100,000, more preferably from 3,000 to 50,000, and especially preferably from 5,000 to 30,000.

In addition, the molecular weight distribution (Mw/Mn) is preferably from 1.0 to 2.5, and more preferably from 1.0 to 2.0 in terms of suppressing nonspecific adsorption and in terms of increasing the activity of a ligand bound to magnetic beads.

The number average molecular weight and the weight average molecular weight mean average molecular weights in terms of polyethylene glycol, which are measured by gel permeation chromatography after releasing chain polymers from magnetic beads by, for example, hydrolysis, and specifically can be measured in accordance with a method as described in the examples mentioned below.

In the present description, the “magnetic beads” mean beads with a magnetic substance. The magnetic beads can be separated by, for example, a magnet without using, for example, a centrifuge, and can be simply or automatically separated from a sample.

The magnetic substance may have any of ferromagnetism, paramagnetism and superparamagnetism and is preferably superparamagnetic in terms of easing separation in a magnetic field and redispersion after removing the magnetic field. Examples of the magnetic substance include metals such as ferrite, iron oxide, iron, manganese oxide, manganese, nickel oxide, nickel, cobalt oxide and cobalt, or alloys. In the magnetic beads of the present invention, even when the magnetic substance has a reducibility as, for example, ferrite, degeneration accompanied by color changes is not brought about.

Specific examples of the magnetic beads include those formed by binding the above chain polymer at least to the surface of any beads in the following (i) to (iv). Porous or non-porous magnetic polymer beads are preferred.

(i) Beads in which magnetic minute particles are dispersed in a continuous phase comprising a non-magnetic substance such as a resin,

(ii) beads in which a secondary agglomerate of magnetic minute particles is constituted as the core and a non-magnetic substance such as a resin is constituted as a shell,

(iii) beads in which mother beads having nuclear beads constituted from a non-magnetic substance such as a resin and a magnetic layer (secondary agglomerate layer) comprising magnetic minute particles provided to the surface of the nuclear beads are constituted as the core, and a non-magnetic layer such as a resin is provided to the outermost layer of the mother beads as a shell (hereinafter, referred to as the outermost layer shell), and

(iv) beads in which magnetic minute particles are dispersed in the holes of porous beads comprising, for example, a resin and silica, in which a non-magnetic layer such as a resin may be provided to the outermost layer of beads as a shell.

The beads in (i) to (iv) are all known and can be produced in accordance with a conventional method.

Examples of the resins in the nuclear beads in (iii) above and the porous beads in (iv) above include resins derived from one or two or more monomers selected from the group consisting of a monofunctional monomer and a cross-linkable monomer.

Examples of the above monofunctional monomer include a monofunctional aromatic vinyl monomer such as styrene, α-methylstyrene and halogenated styrene; and a monofunctional (meth)acrylate monomer such as methyl (meth)acrylate, ethyl (meth)acrylate, stearyl (meth)acrylate, cyclohexyl (meth)acrylate and isobornyl (meth)acrylate.

Examples of the above multifunctional monomers include, for example, multifunctional aromatic vinyl monomers such as divinylbenzene; multifunctional (meth)acrylate monomers such as ethyleneglycol di(meth)acrylate, trimethylolpropane tri(meth)acrylate, dipentaerythritol hexa(meth)acrylate and allyl (meth)acrylate; and conjugated diolefins such as butadiene and isoprene.

In addition, as the resins in (i) and (ii) above as well as the resins in the outermost layer shell in (iii) and (iv) above, a resin having one or two or more functional groups selected from the group consisting of a glycidyl group, an amino group and a hydroxy group at least on the surface is preferred. The above functional groups may be introduced by the chemical modification of the resin surface, or by polymerization of monomer composition at least comprising one or two or more monomers having the above functional group. Examples of the above chemical modification include the formation of a hydroxy group by hydrolysis of a glycidyl group, and the formation of an amino group by reduction of a nitro group. As the above monomer composition having a functional group, monomer composition at least comprising a glycidyl group-containing monomer is more preferred (hereinafter, beads in which the resin in the above outermost layer shell is a resin formed by monomer composition at least comprising a glycidyl group-containing monomer are also referred to as a glycidyl group-containing magnetic beads). One or two or more monomers selected from the group consisting of the above mono functional monomer and cross-linkable monomer may be further contained.

Examples of the glycidyl group-containing monomer include, for example, glycidyl (meth)acrylate and allyl glycidyl ether. Examples of the amino group-containing monomer include, for example, 2-aminoethyl (meth)acrylate. Examples of the hydroxy group-containing monomer include, for example, 1,4-cyclohexane dimethanol mono(meth)acrylate.

In addition, the average particle diameter (volume average particle diameter) of the magnetic beads of the present invention is preferably from 0.1 to 500 more preferably from 0.2 to and further preferably from 0.3 to 10 Within such a range, the magnetic collection speed becomes faster and handling properties are improved, and also the amount of bound ligand becomes larger, and detection sensitivity becomes higher. In addition, the coefficient of variation of the average particle diameter is only required to be about 20% or less.

The specific surface area is only required to be about from 1.0 to 2.0 m²/g.

The above average particle diameter and specific surface area can be measured by, for example, laser diffraction scattering bead size distribution measurement.

### <Method for Producing Magnetic Beads>

The magnetic beads of the present invention can be produced by properly combining conventional methods, and are preferably produced by a method comprising (Step 1) a step of preparing a magnetic bead material having a polymerization initiating group at least on the surface (hereinafter, also referred to as polymerization initiating group-containing beads), (Step 2) a step of polymerizing a hydrophilic monomer from the polymerization initiating group, and (Step 3) a step of allowing a compound having a primary or secondary amino group to react with the end of a chain polymer formed on the surface of the magnetic bead material in the step 2. According to such a method, the density of the chain polymer occupying the surface of magnetic beads can be highly increased, and further the intended magnetic beads can be simply obtained.

When, as a compound having a primary or secondary amino group used in the step 3, a compound which does not comprise a reactive functional group except a primary or secondary amino group is used, the magnetic beads of the present invention are obtained by further undergoing the following step 4:

(Step 4) a step of introducing a reactive functional group into the end structure added in the step 3 by an addition reaction, a substitution reaction or a condensation reaction.

Meanwhile, when, as a compound having a primary or secondary amino group used in the step 3, a compound comprising, in addition to a primary or secondary amino group, further a reactive functional group is used, the magnetic beads of the present invention are obtained without the step 4.

(Step 1)

Polymerization initiating group-containing beads can be obtained, for example, by bringing a compound having a polymerization initiating group into contact with magnetic beads having one or two or more groups selected from the group consisting of a hydroxy group, an amino group, an epoxy group and a carboxy group (hereinafter, these are collectively referred to as hydroxy group etc.) at least on the surface (hereinafter, also referred to as hydroxy group etc.-containing beads) to convert a hydrogen atom contained in the above hydroxy group etc. to the polymerization initiating group (hereinafter, this reaction is also referred to as a polymerization initiating group introducing reaction). Among the above hydroxy group etc.-containing beads, magnetic beads having hydroxy groups at least on the surface can be obtained, for example, by bringing the above glycidyl group-containing magnetic beads into contact with an acid such as inorganic acid or an organic acid to conduct a ring-opening of the glycidyl group.

Polymerization initiating group-containing beads can be also obtained by polymerizing monomer composition comprising a monomer having a polymerization initiating group. Examples of the monomer having a polymerization initiating group include, for example, 2-(2-bromoisobutyryloxy)ethyl methacrylate.

As the above compound having a polymerization initiating group, a compound having a polymerization initiating group capable of living polymerization is preferred, a compound having a living radical polymerization initiating group is more preferred, a compound having an atom transfer radical polymerization initiating group and a compound having a reversible addition-fragmentation chain transfer polymerization initiating group are further preferred, and a compound having an atom transfer radical polymerization initiating group is especially preferred. Examples of the compound having an atom transfer radical polymerization initiating group include, for example, 2-bromoisobutyryl bromide, 4-(bromomethyl)benzoic acid, ethyl 2-bromoisobutyrate, 2-bromopropionyl bromide and tosyl chloride.

In the polymerization initiating group introducing reaction, a total amount of compound having a polymerization initiating group used is normally about from 5 to 10,000 molar equivalents and preferably about from 10 to 5,000 molar equivalents with respect to the hydroxy group etc.-containing beads.

It is preferred that the polymerization initiating group introducing reaction be carried out in the presence of a basic catalyst such as triethylamine, N,N-dimethyl-4-aminopyridine, diisopropylethylamine or pyridine. One of these basic catalysts may be used alone or two or more of these basic catalysts may be used in combination.

The total amount of basic catalyst used is normally about from 1 to 10 molar equivalents and preferably about from 1 to 5 molar equivalents with respect to a compound having a polymerization initiating group.

It is also preferred that the polymerization initiating group introducing reaction be carried out in the presence of a solvent. Examples of solvents include ether solvents such as tetrahydrofuran, 1,4-dioxane, 1,3-dioxane and 1,3-dioxolane; and aprotic solvents such as dimethylformamide and dimethylsulfoxide, and one of these solvents can be used alone or two or more of these solvents can be used in combination.

In addition, the reaction time of the polymerization initiating group introducing reaction is normally about from 30 minutes to 24 hours, and the reaction temperature may be properly selected from the boiling point of a solvent or lower.

(Step 2)

The polymerization method for the polymerization reaction in the step 2 may be selected depending on a kind of polymerization initiating group, and living polymerization is preferred, living radical polymerization is more preferred, atom transfer radical polymerization (ATRP polymerization) and reversible addition-fragmentation chain transfer polymerization (RAFT polymerization) are further preferred, and atom transfer radical polymerization is especially preferred in terms of simply and easily obtaining a target product. By polymerization by atom transfer radical polymerization, a chain polymer can be allowed to simply bind to a wide variety of beads, and further biocompatibility, high compressive elasticity, low frictional characteristics and size exclusion characteristics are imparted to magnetic beads thus obtained, and the density of chain polymer occupying the surface of magnetic beads is highly increased, and thus nonspecific adsorption is suppressed.

Examples of the hydrophilic monomer are, for example, a monomer having a hydroxy group, a monomer having a polyoxyethylene group, a monomer having a group having a zwitterion, a monomer having a phosphate group, and further monomers showing hydrophilicity such as dimethyl (meth)acrylamide, dimethylaminopropyl (meth)acrylamide, isopropyl (meth)acrylamide and diethyl (meth)acrylamide.

Examples of the monomer having a hydroxy group are, for example, 2-hydroxyethyl (meth)acrylate, 2-hydroxyethyl (meth)acrylamide, 2-hydroxypropyl (meth)acrylate, 2-hydroxypropyl (meth)acrylamide, 2-hydroxybutyl (meth)acrylate, 2-hydroxybutyl (meth)acrylamide, glycerol 1-(meth)acrylate and glycerol 1-(meth)acrylamide. Examples of the monomer having a polyoxyethylene group are, for example, methoxypolyethylene glycol mono(meth)acrylate and methoxypolyethylene glycol mono(meth)acrylamide. Examples of the monomer having a group having a zwitterion are, for example, [2-((meth)acryloyloxy)ethyl](carboxylatomethyl)dimethylaminium, [2-((meth)acryloyloxy)ethyl]dimethyl-(3-sulfopropyl) ammonium hydroxide, and O-[2-((meth)acryloyloxy) ethoxy(oxylato)phosphinyl]choline. Examples of the monomer having a phosphate group are, for example, 2-phosphoric ethyl (meth)acrylate and 2-phosphoric ethyl (meth)acrylamide.

One of these monomers may be used alone or two or more of these monomers may be used in combination.

The total amount of hydrophilic monomer used is normally about from 5 to 10,000 molar equivalents and preferably about from 10 to 5,000 molar equivalents with respect to a polymerization initiating group binding to the surface of a magnetic bead material.

When the polymerization reaction in the step 2 is carried out by atom transfer radical polymerization, it is preferred that the reaction be carried out in the presence of a transition metal compound and a ligand in terms of reaction efficiency.

As the transition metal compound, a copper compound is preferred. Examples of the copper compound include, for example, halogenated copper such as copper(I) bromide, copper(II) bromide, copper(I) chloride and copper(II) chloride, and further copper(I) triflate and copper(II) triflate. One of these compounds may be used alone or two or more of these compounds may be used in combination. The total amount of transition metal compound used is normally about from 1 to 10,000 ppm in the reaction system.

As the ligand, a ligand comprising two or more nitrogen atoms in the same molecule is preferred. Examples of the ligand comprising two or more nitrogen atoms in the same molecule include, for example, tris(2-pyridylmethyl)amine, bipyridine, bipyridine derivatives and tris[2-(dimethylamino)ethyl]amine. One of these ligands may be used alone or two or more of these ligands may be used in combination.

The total amount of ligand used is normally about from 0.5 to 10 times by mass with respect to a transition metal compound.

It is also preferred that the polymerization reaction in the step 2 be carried out in the presence of a reducing agent and a solvent in terms of reaction efficiency.

Examples of reducing agents include, for example, ascorbic acid, glucose, hydrazine and copper, and one of these reducing agents can be used alone or two or more of these reducing agents can be used in combination.

Examples of solvents include, for example, water; amide solvents such as dimethylformamide; alcohol solvents such as methanol and ethanol and one of these solvents can be used alone or two or more of these solvents can be used in combination.

The pH in the reaction system of the polymerization reaction is preferably from 3 to 10. The reaction time of the polymerization reaction is normally about from 30 minutes to 12 hours, and the reaction temperature may be properly selected from the boiling point of a solvent or lower. The polymerization reaction proceeds even under mild conditions of about from 25 to 60° C.

(Steps 3 and 4)

The step 3 is the step of, by allowing a compound having a primary or secondary amino group (hereinafter, also referred to as amine compound) to react with the end (e.g. end halogen atom) of a chain polymer formed on the surface of the magnetic bead material in the step 2, introducing an imino group or an N-substituted imino group in the end part of the chain polymer.

Examples of the above amine compound include, for example, a compound having two or more amino groups in a molecule, a compound having an amino group and a hydroxy group in a molecule, and further a compound having an amino group and a carboxy group, and a compound having an amino group and an epoxy group. When a compound having two or more amino groups in a molecule, a compound having an amino group and a carboxy group, and a compound having an amino group and an epoxy group, for example, are used as the amine compound, the magnetic beads of the present invention are obtained without the step 4.

Examples of the compound having two or more amino groups in a molecule include, for example, ethylenediamine, 1,2-bis(2-aminoethoxy) ethane, diethylene glycol bis(3-aminopropyl) ether, cadaverine, hexamethylenediamine, tris(2-aminoethyl)amine and polyethyleneimine. Examples of the compound having an amino group and a hydroxy group in a molecule include, for example, ethanolamine, 3-amino-1-propanol, 4-amino-1-butanol, 5-amino-1-pentanol, 6-amino-1-hexanol, 8-amino-1-octanol, 10-amino-1-decanol and 12-amino-1-dodecanol. Examples of the compound having an amino group and a carboxy group include amino acids. One of these compounds may be used alone or two or more of these compounds may be used in combination.

The total amount of amine compound used is normally about from 0.1 to 2,000 molar equivalents and preferably from 1 to 1,000 molar equivalents with respect to chain polymers formed in the step 2.

The step 4 is the step of introducing a reactive functional group into the end structure (e.g. the end structure comprising a hydroxy group) added in the step 3 by an addition reaction, a substitution reaction or a condensation reaction.

Examples of the method for introducing a carboxy group as a reactive functional group include, for example, a method in which the above hydroxy group is subjected to an addition reaction with carboxylic anhydride, and examples of the method for introducing a tosyl group as a reactive functional group include, for example, a method in which tosyl chloride is allowed to react with the above hydroxy group.

Examples of carboxylic anhydride include, for example, succinic anhydride, maleic anhydride, glutaric anhydride, phthalic anhydride and hexahydrophthalic anhydride.

By using a compound having two or more amino groups in a molecule and a compound having an amino group and an epoxy group in the step 3, the reactive functional group introduced in the end of a chain polymer may be converted from an amino group or an epoxy group to a carboxy group and a tosyl group. This conversion may be carried out, for example, by a method in which a compound having two or more carboxy groups in a molecule or tosyl chloride is allowed to react with the above amino group, and a method in which mercaptopropionic acid is allowed to react with the above epoxy group.

The total amount of compound used to introduce a reactive functional group in the step 4 is normally about from 1 to 1,000 molar equivalents and preferably from 5 to 500 molar equivalents with respect to the end structure added in the step 3.

It is preferred that the above steps 3 and 4 be carried out in the presence of the same basic catalyst and solvent as in the step 1 in terms of efficiently obtaining the intended magnetic beads. The total amount of basic catalyst used is normally about from 0.001 to 1 molar equivalent and preferably about from 0.01 to 1 molar equivalent with respect to a compound to be introduced.

The reaction times in the steps 3 and 4 are each normally about from 30 minutes to 24 hours, and the reaction temperature may be properly selected from the boiling point of a solvent or lower.

In the magnetic beads of the present invention obtained as above, a target substance and impurities in a sample are hard to adsorb on the surface, and nonspecific adsorption is suppressed, and degeneration accompanied by color changes is not brought about. Further, since a sufficient amount of ligand to detect and separate a target substance from a sample can be allowed to bind thereto, an enhancement in sensitivity and a decrease in noise in detection and separation of a target substance, and an increase in purity can be achieved by using the magnetic beads of the present invention.

Therefore, affinity supports obtained from the magnetic beads of the present invention can be widely used, for example, for in vitro diagnoses and research in the biochemical field, including, for example, immunoassay using antigen-antibody reactions such as enzyme immunoassay, radioimmunoassay and chemiluminescence immunoassay; the detection of, for example, proteins and nucleic acids; bioseparation of bio-related materials such as cells, proteins and nucleic acids; drug seeking; and biosensors. The magnetic beads of the present invention are especially suitable for use for immunoassay or detecting nucleic acids.

### <Ligand-Binding Beads>

The ligand-binding beads of the present invention are formed by binding a ligand to the magnetic beads of the present invention.

The above ligand is only required to be a molecule which binds to a target substance, and examples thereof include, for example, antibodies; antigens; nucleic acids such as DNA and RNA; nucleotides; nucleosides; proteins such as Protein A, Protein G, (strept)avidin, enzymes and lectins; peptides such as insulin; amino acids; saccharides or polysaccharides such as heparin; lipids; vitamins such as biotin; medicine; substrates; hormones; and neurotransmitters.

Among these ligands, antibodies and antigens are preferred in terms of obtaining ligand-binding beads suitable for, for example, diagnostic agents. The antibodies and antigens are only required to bind to a target substance, and examples thereof include antibodies for coagulation fibrinolysis tests such as anti-antiplasmin antibody, anti-D dimer antibody, anti-FDP antibody, anti-tPA antibody, anti-thrombin-antithrombin complex antibody and anti-FPA antibody or antigens thereof; antibodies for tumor tests such as anti-BFP antibody, anti-CEA antibody, anti-AFP antibody, anti-TSH antibody, anti-ferritin antibody and anti-CA19-9 antibody or antigens thereof; antibodies for serum protein tests such as anti-apolipoprotein antibody, anti-β2-microglobulin antibody, anti-α1-microglobulin antibody, anti-immunoglobulin antibody and anti-CRP antibody or antigens thereof; antibodies for endocrine function tests such as anti-HCG antibody or antigens thereof; antibodies for medicine analyses such as anti-digoxin antibody and anti-lidocaine antibody or antigens thereof; antigens for infectious disease tests such as HBs antigen, HCV antigen, HIV-1 antigen, HIV-2 antigen, HTLV-1 antigen, mycoplasma antigen, toxoplasma antigen and streptolysin O antigen or antibodies thereof; and antigens for autoimmune tests such as DNA antigen and heat-aggregated human IgG or antibodies thereof. The antibodies may be polyclonal antibodies or monoclonal antibodies.

The binding of ligands may be carried out in accordance with a conventional method, and is preferably carried out by a covalent binding method. For example, when a reactive functional group is a carboxy group and a ligand has an amino group, binding may be carried out using a dehydration-condensation agent.

The ligand-binding beads of the present invention can be widely used for, for example, in vitro diagnosis and research in the biochemical field. The ligand-binding beads of the present invention are especially suitable for use for immunoassay or detecting nucleic acids.

### <Method for Detecting or Separating Target Substance>

The method for detecting or separating a target substance in a sample according to the present invention is characterized by using the ligand-binding beads of the present invention.

The target substance is not limited as long as it binds to a ligand, and specific examples thereof include antigens; antibodies such as monoclonal antibodies and polyclonal antibodies; cells (normal cells, and cancer cells such as colon cancer cells and circulating cancer cells in blood); nucleic acids such as DNA and RNA; bio-related materials such as proteins, peptides, amino acids, saccharides, polysaccharides, lipids and vitamins, and the target substances may be small molecular compounds such as a drug as a target for drug discovery and biotin. The target substance may be labeled by, for example, a fluorescent substance.

The sample is not limited as long as it comprises the above target substance or has the possibility of comprising a target substance, and are specifically, for example, blood, blood plasma, blood serum and buffer solutions containing a target substance.

The method of detection or separation according to the present invention may be carried out in accordance with a conventional method except that the ligand-binding beads of the present invention are used. Examples thereof include a method comprising a step of bringing the ligand-binding beads of the present invention into contact with a sample comprising a target substance by, for example, mixing (contact step), and a step of separating the ligand-binding beads which have grasped the target substance from the sample using, for example, a magnet (separation step). After such a separation step, a step of detecting the target substance or a step of dissociating the ligand and the target substance may be included.

### EXAMPLES

The present invention will now be described in detail by way of examples thereof. It should be noted, however, that the present invention is not limited to these examples. Each analysis condition in the examples is as described below.

### <Measurement of Molecular Weight of Chain Polymer>

The molecular weight of chain polymer was measured after releasing chain polymers from beads by hydrolysis using an aqueous solution of sodium hydroxide.

That is, 1 g of beads was dispersed in 4 g of an aqueous solution of sodium hydroxide (1 N, pH 14), and the obtained mixture was stirred at 25° C. for 3 hours to release chain polymers from beads. The beads were separated using magnetism, and the supernatant in which the chain polymers had been dissolved was collected. Next, to this chain polymer solution was added 1 M hydrochloric acid until the pH of the solution became 7 to neutralize the solution. In order to be used in calculating the weight of chain polymer, the weight of sodium chloride thus produced was calculated from the weight of 1 M hydrochloric acid added. The solution after neutralization was freeze-dried to obtain the chain polymer comprising sodium chloride as powders. In order to be used in calculating the weight of chain polymer, the weight of powder was measured.

The above powders were used as a test specimen, and the Mn and Mw of chain polymer formed on the surface of beads were measured under the following conditions by gel permeation chromatography (GPC) using TSKgel G3000PWXL column manufactured by Tosoh Corporation and ChromNAV chromatography data station program manufactured JASCO International Co., Ltd.

(Measurement Conditions)

Flow rate: 0.8 mL/min

Eluting solvent: 0.2 M sodium phosphate buffer (pH 7.0)

Column temperature: 25° C.

Reference material: TSKgel standard Poly(ethylene oxide) SE-kit manufactured by Tosoh Corporation and Polyethylene Glycol 4,000 manufactured by Wako Pure Chemical Industries, Ltd.

### <Polymer Density of Chain Polymer Occupying Surface of Beads>

The polymer density was calculated from the weight of chain polymer released from beads, the number average molecular weight of chain polymer and the surface area of beads by the following formula.

[Density of chain polymer occupying surface of beads (chain polymers/nm²)]=[Number of chain polymers binding to 1 g of beads (chain polymers)]/[Total surface area per g of beads (nm²)]

Note that the methods for calculating the number of chain polymers binding to 1 g of beads and the total surface area per g of beads are as described below.

(Number of Chain Polymers Binding to 1 g of Beads)

The weight of chain polymer binding to 1 g of beads was calculated by the following formula (α), and using the obtained value, the number of chain polymers binding to 1 g of beads was calculated by the following formulae (β) and (γ):

weight of chain polymer binding to 1 g of beads (mg)=weight of powder after freeze-drying (mg)−weight of sodium chloride (mg), (α):

number of chain polymers binding to 1 g of beads (mol)={weight of chain polymer binding to 1 g of beads (mg)/number average molecular weight of chain polymer (g/mol)}/1000, and (β):

number of chain polymers binding to 1 g of beads (chain polymers)=number of chain polymers binding to 1 g of beads (mol)×6.02×10²³(Avogadro's number). (γ):

(Total Surface Area Per g of Beads)

The total surface area was calculated by the following formulae (δ) to (θ). The specific gravity of beads in the formula (ε) was calculated from the specific gravity of a polymer, the specific gravity of a magnetic substance, and the ratio of the polymer and the magnetic substance occupying beads:

volume per bead (μm³)=4/3×π×{volume average radius of beads (μm)}³, (δ):

mass per bead (g)=volume per bead (μm³)×specific gravity of beads (g/μm³), (ε):

number of beads per g of beads (beads)=1 g/mass per bead (g), (ζ):

surface area per bead (nm²)=4×π×{radius of bead (nm)}², and (η):

total surface area per g of beads (nm²)=surface area per bead (nm²)×number of beads per g of beads (beads). (θ):

### <Content of Reactive Functional Group>

The content of reactive functional group per g of solid content in magnetic beads was obtained by measuring the content of reactive functional group (carboxy group) contained in chain polymers released from beads using conductometry (Metrohm, 794 Basic Titrino).

### <Volume Average Particle Diameter>

The volume average particle diameter of the beads was measured by a laser diffraction scattering bead size distribution measuring device (Beckman Coulter LS13 320).

### Synthetic Example 1

### Synthesis of Magnetic Beads Having Hydroxy Group on Surface

With 20 g of a 1 mass % aqueous solution of dodecyl sodium sulfate, 2 g of a 75% solution of di(3,5,5-trimethyl hexanoyl)peroxide (“PEROYL 355-75 (S)” manufactured by NOF Corporation) was mixed, and the obtained mixture was finely emulsified by an ultrasonic disperser. This was put into a reactor comprising 13 g of polystyrene beads (number average particle diameter: 0.77 and 41 g of water, and the mixture was stirred at 25° C. for 12 hours.

Next, 96 g of styrene and 4 g of divinyl benzene were emulsified with 400 g of a 0.1 mass % aqueous solution of dodecyl sodium sulfate in another container, and this was put into the above reactor, and the mixture was stirred at 40° C. for 2 hours, followed by raising the temperature to 75° C., and polymerization was carried out for 8 hours. After cooling to room temperature, only beads taken out by centrifugation were washed with water and dried. These beads were used as nuclear beads (number average particle diameter: 1.5 μm).

Next, acetone was added to oily magnetic fluid (“EXP series, EMG”, manufactured by Ferrotec Corporation) in another container to precipitate and deposit beads, and these were then dried to obtain ferrite magnetic minute particles having the surface hydrophobized (average primary particle diameter: 0.01

Next, 15 g of the above nuclear beads and 15 g of the above hydrophobized magnetic minute particles were mixed well by a mixer, and this mixture was treated using an NHS-0 type hybridization system manufactured by Nara Machinery Co., Ltd. at a circumferential velocity of wings (impeller) of 100 m/sec (16,200 rpm) for 5 minutes to obtain mother beads having a magnetic layer comprising the magnetic minute particles on the surface (number average particle diameter: 2.0 μm).

Next, 250 g of a 0.50 mass % aqueous solution of dodecyl sodium sulfate was charged into a 500 mL separable flask, and then 10 g of the above mother beads having a magnetic layer were added, and the obtained mixture was dispersed by a homogenizer and then heated to 60° C., and the temperature was maintained.

Next, 75 g of a 0.50 mass % aqueous solution of dodecyl sodium sulfate, 13.5 g of methyl methacrylate (hereinafter, referred to as “MMA”), 1.5 g of trimethylolpropane trimethacrylate (hereinafter, referred to as “TMP”), and 0.3 g of a 75% solution of di(3,5,5-trimethyl hexanoyl)peroxide (“PEROYL 355-75 (S)” manufactured by NOF Corporation) were put into another container and dispersed to obtain a pre-emulsion. The total amount of this pre-emulsion was added dropwise to the above 500 mL separable flask maintained at 60° C. over a period of two hours. After a dropwise addition, this was maintained at 60° C. and the mixture was stirred for an hour.

After that, 37.5 g of a 0.50 mass % aqueous solution of dodecyl sodium sulfate, 6.56 g of glycidyl methacrylate (hereinafter, referred to as “GMA”), 0.94 g of TMP, and 0.15 g of a 75% solution of di(3,5,5-trimethyl hexanoyl)peroxide (“PEROYL 355-75 (S)” manufactured by NOF Corporation) were put into another container and dispersed to obtain a pre-emulsion. The total amount of this pre-emulsion was added dropwise to the above 500 mL separable flask maintained at 60° C. over a period of an hour and 20 minutes. After that, the temperature was elevated to 75° C. and polymerization was then continued for another two hours, and the reaction was completed. Subsequently, 10 mL of a 1 mol/L aqueous solution of sulfuric acid was put into this 500 mL separable flask and the mixture was stirred at 60° C. for 6 hours. Next, the beads in the above 500 mL separable flask were separated using magnetism and then repeatedly washed with distilled water.

As described above, the magnetic beads having hydroxy groups on the surface were obtained.

### Synthetic Example 2

### Synthesis of Magnetic Beads Having Atom Transfer Radical Polymerization Initiating Group on Surface (1)

Into a flask, 10 g of magnetic beads having hydroxy groups on the surface, obtained in Synthetic Example 1, were charged, and 32 mL of dehydrated tetrahydrofuran and 7.5 mL of triethylamine were added under a nitrogen flow and the mixture was stirred. This flask was soaked in an ice bath, and thereto was added 6.3 mL of 2-bromoisobutyryl bromide dropwise over a period of 30 minutes. After a reaction at room temperature for 6 hours, the beads in the flask were separated using magnetism, and the beads were then redispersed in acetone. The magnetic separation and redispersion were carried out another several times, and the beads were then dispersed in a 0.10 mass % aqueous solution of dodecyl sodium sulfate. Br contained in the atom transfer radical polymerization initiating group (2-bromoisobutyryl group) was detected by a fluorescent X-ray analysis.

As described above, the magnetic beads having the atom transfer radical polymerization initiating group (2-bromoisobutyryl group) on the surface were obtained. These beads were used as beads (A).

### Synthetic Example 3

### Synthesis of Magnetic Beads Having Atom Transfer Radical Polymerization Initiating Group on Surface (2)

Into a flask, 10 g of magnetic beads having hydroxy groups on the surface, obtained in Synthetic Example 1, were charged, and 32 mL of dehydrated tetrahydrofuran and 0.4 mL of triethylamine were added under a nitrogen flow and the mixture was stirred. This flask was soaked in an ice bath, and 0.2 mL of 2-bromoisobutyryl bromide was added. After a reaction at room temperature for 6 hours, the beads in the flask were separated using magnetism, and the beads were then redispersed in acetone. The magnetic separation and redispersion were carried out another several times, and the beads were then dispersed in a 0.10 mass % aqueous solution of dodecyl sodium sulfate. Br contained in the atom transfer radical polymerization initiating group (2-bromoisobutyryl group) was detected by a fluorescent X-ray analysis.

As described above, the magnetic beads having the atom transfer radical polymerization initiating group (2-bromoisobutyryl group) on the surface were obtained. These beads are used as beads (B).

### Synthetic Example 4

### Synthesis of Magnetic Beads Having Atom Transfer Radical Polymerization Initiating Group on Surface (3)

Into a flask, 10 g of magnetic beads having hydroxy groups on the surface, obtained in Synthetic Example 1, were charged, and 32 mL of dehydrated tetrahydrofuran and 0.2 mL of triethylamine were added under a nitrogen flow and the mixture was stirred. This flask was soaked in an ice bath, and 0.1 mL of 2-bromoisobutyryl bromide was added dropwise over a period of 30 minutes. After a reaction at room temperature for 6 hours, the beads in the flask were separated using magnetism, and the beads were then redispersed in acetone. The magnetic separation and redispersion were carried out another several times, and the beads were then dispersed in a 0.10 mass aqueous solution of dodecyl sodium sulfate. Br contained in the atom transfer radical polymerization initiating group (2-bromoisobutyryl group) was detected by a fluorescent X-ray analysis.

As described above, the magnetic beads having the atom transfer radical polymerization initiating group (2-bromoisobutyryl group) on the surface were obtained. These beads are used as beads (C).

### Example 1

(1) The chain polymer propagation reaction was carried out in accordance with the following synthetic route.

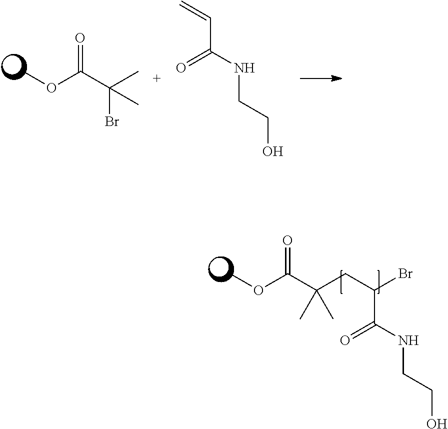

That is, 2 g of the beads (A) obtained in Synthetic Example 2 were dispersed in 6 mL of a sodium phosphate buffer (50 mM, pH 7.8), and 0.5 g of 2-hydroxyethyl acrylamide (hereinafter, referred to as “HEAA”) and 0.40 mL of a mixed aqueous solution of 0.05 mol/L tris(2-pyridylmethyl)amine and 0.05 mol/L copper(II) bromide were added thereto.

Subsequently, 1.0 mL of a 0.2 mol/L aqueous solution of L-ascorbic acid was added thereto and a stopper was sealed to start the reaction. After stirring at 45° C. for 4 hours, the stopper was opened to stop the reaction by an exposure to air. The beads were separated using magnetism to remove, for example, unreacted monomers and catalysts.

(2) A group comprising a carboxy group was introduced in the end of a polymer chain through an imino group in accordance with the following synthetic route.

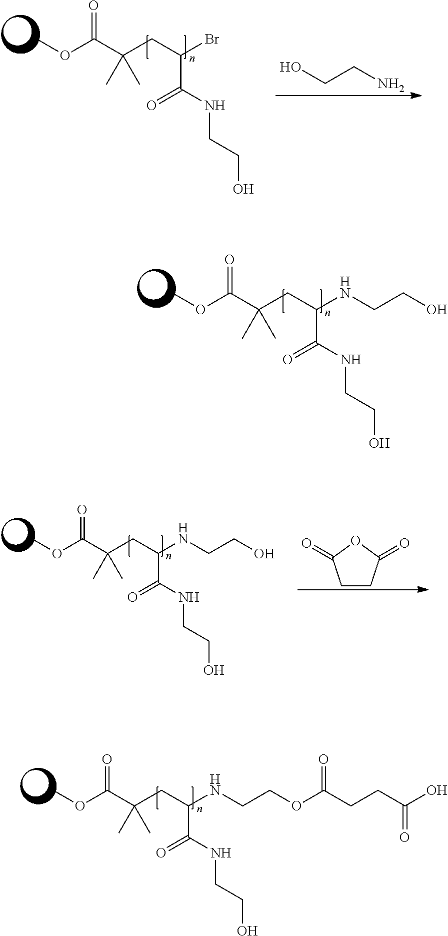

That is, the beads obtained above were dispersed in 6 mL of dimethylsulfoxide, and 1 mL of ethanolamine and 0.2 mL of triethylamine were added. After stirring at 45° C. for 5 hours, the beads were separated using magnetism to remove unreacted compounds.

Next, the obtained beads were dispersed in 4.8 mL of 1,3-dioxolane, and a solution in which 0.2 mL of triethylamine and 2 g of succinic anhydride were dissolved in 4.8 mL of 1, 3-dioxolane was added. After a reaction at 25° C. for 4 hours, the beads were separated using magnetism and the beads were dispersed in water.

As described above, the magnetic beads 1 in which a chain polymer derived from HEAA binds to the surface, and which have a group comprising a carboxy group at the end of such a chain polymer through an imino group (volume average particle diameter: 3.0 μm) were obtained. The Mn, Mw and Mw/Mn of chain polymer binding to the magnetic beads 1 and the polymer density were measured. In addition, the content of reactive functional group (carboxy group) was measured. The measurement results are shown in Table 1.

### Example 2

By the same operation as in Example 1 except that HEAR was changed to [2-(methacryloyloxy)ethyl](carboxylatomethyl)dimethylaminium (hereinafter, referred to as “CBMA”), the magnetic beads 2 in which a chain polymer derived from CBMA binds to the surface, and which have a group comprising a carboxy group at the end of such a chain polymer through an imino group (volume average particle diameter: 3.0 μm) were obtained.

The results of each measurement carried out in the same manner as in Example 1 are shown in Table 1.

### Example 3

By the same operation as in Example 1 except that HEAR was changed to methoxypolyethylene glycol (9) monomethacrylate (M-90G manufactured by Shin-Nakamura Chemical Co., Ltd. (hereinafter, referred to as “MPEGM”)), the magnetic beads 3 in which a chain polymer derived from MPEGM binds to the surface, and which have a group comprising a carboxy group at the end of such a chain polymer through an imino group (volume average particle diameter: 3.0 μm) were obtained.

The results of each measurement carried out in the same manner as in Example 1 are shown in Table 1.

### Example 4

The magnetic beads 4 in which a chain polymer derived from HEAA binds to the surface, and which have a group comprising a carboxy group at the end of such a chain polymer through an imino group (volume average particle diameter: 3.0 μm) were obtained by the same operation as in Example 1 except that the beads (A) were changed to the beads (B) obtained in Synthetic Example 3.

The results of each measurement carried out in the same manner as in Example 1 are shown in Table 1.

### Comparative Example 1

(1) The chain polymer propagation reaction was carried out in accordance with the following synthetic route.

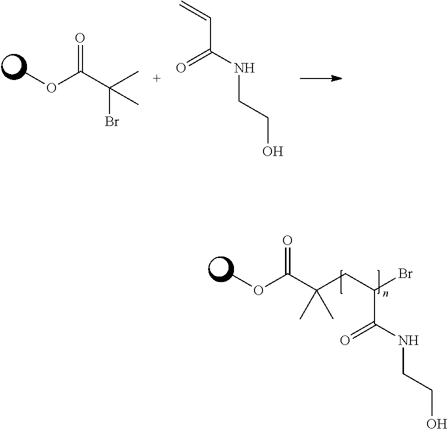

That is, 2 g of the beads (A) obtained in Synthetic Example 2 was dispersed in 6 mL of a sodium phosphate buffer (50 mM, pH 7.8), and 0.5 g of HEAR and 0.40 mL of a mixed aqueous solution of 0.05 mol/L tris(2-pyridylmethyl)amine and 0.05 mol/L copper (II) bromide were added thereto. Subsequently, 1.0 mL of a 0.2 mol/L aqueous solution of L-ascorbic acid was added thereto and a stopper was sealed to start the reaction. After stirring at 45° C. for 4 hours, the stopper was opened to stop the reaction by an exposure to air. The beads were separated using magnetism to remove, for example, unreacted monomers and catalysts.

(2) A group comprising a carboxy group was introduced in the end of a polymer chain through a thio group in accordance with the following synthetic route.

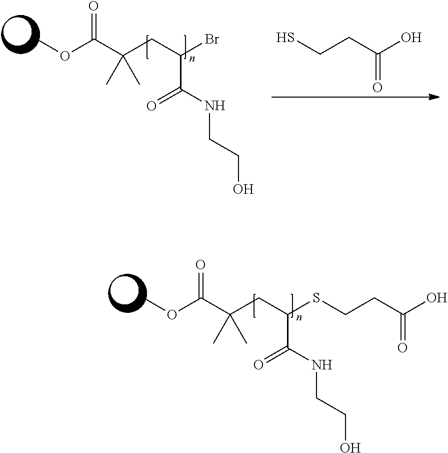

That is, the beads obtained above were dispersed in 6 mL of dimethylsulfoxide, and 1 mL of mercaptopropionic acid was added. After stirring at 45° C. for 5 hours, the beads were separated using magnetism to remove unreacted compounds.

As described above, the magnetic beads 5 in which a chain polymer derived from HEAA binds to the surface, and which have a group comprising a carboxy group at the end of such a chain polymer through an thio group (volume average particle diameter: 3.0 μm) were obtained. The Mn, Mw and Mw/Mn of chain polymer binding to the magnetic beads 5 and the polymer density were measured. The measurement results are shown in Table 1.

### Comparative Example 2

The magnetic beads 6 in which a chain polymer derived from CBMA binds to the surface, and which have a group comprising a carboxy group at the end of such a chain polymer through an imino group (volume average particle diameter: 3.0 μm) were obtained by the same operation as in Example 2 except that the beads (A) were changed to the beads (C) obtained in Synthetic Example 4.

The results of each measurement carried out in the same manner as in Example 1 are shown in Table 1.

### Comparative Example 3

In 4.8 mL of 1,3-dioxolane, 1 g of magnetic beads having hydroxy groups on the surface obtained in Synthetic Example 1 were dispersed, and a solution in which 0.2 mL of triethylamine and 0.08 g of succinic anhydride were dissolved in 4.8 mL of 1, 3-dioxolane was added. After a reaction at 25° C. for 4 hours, the beads were separated using magnetism and the beads were dispersed in water. As described above, the magnetic beads 7 containing a reactive functional group, which does not have a chain polymer (volume average particle diameter: 3.0 μm) were obtained. In the magnetic beads 7, the content of reactive functional group (carboxy group) was measured. The measurement results are shown in Table 1.

### Test Example 1

In magnetic beads obtained in each of Examples as well as Comparative Examples 1 and 2, the color of magnetic beads before converting the end structure of a chain polymer (magnetic beads to which a chain polymer having bromine at the end binds) was visually compared to observe color changes. When color changes occurred as compared to before converting the end structure, it can be said that degeneration of beads occurred.

### Test Example 2

In 2 mL of water, 1 mg of magnetic beads obtained in each Example as well as Comparative Examples 2 and 3 were dispersed. This water dispersion was charged into an Eppendorf tube, and the beads were separated using magnetism to remove a supernatant. Next, to the beads, 100 μL of a Jurkat cell disrupted liquid (comprising 100 μg of protein impurities) was added and incubated for 30 minutes. The beads were then separated using magnetism to remove a supernatant, and the beads were washed 5 times with a TBS-T (0.05 mass % Tween 20) buffer.

Further, the beads were separated using magnetism to remove a supernatant, and an aqueous solution of sodium dodecylbenzenesulfonate (0.5 mass %) was then added to detach nonspecifically adsorbing protein impurities from the beads. This detaching solution was subjected to SDS-polyacrylamide gel electrophoresis, and the gel was subjected to CBB stain to visually observe the amount of protein nonspecifically adsorbing to the beads, and evaluation was carried out by the following criteria.

It is evaluated that the beads with a less amount of protein are good beads with less nonspecific adsorption. The evaluation results are shown in Table 1.

(Evaluation Criteria)

A: Protein adsorption is not observed, very good,

B: protein adsorption is hardly observed, good, and

C: protein adsorption is clearly observed, not good.

### Test Example 3

In 2 mL of water, 1 mg of magnetic beads obtained in each Example as well as Comparative Examples 2 and 3 were dispersed. This water dispersion was charged into an Eppendorf tube, and the beads were separated using magnetism to remove a supernatant. Next, the beads were dispersed in 990 μL of a MES buffer (100 mM, pH 5.0), and 10 μL of 1-ethyl-3-(3-dimethylaminopropyl)carbodiimide hydrochloride (10 mg/mL) was added and the obtained mixture was incubated at room temperature for 30 minutes. The beads were separated using magnetism to remove a supernatant and dispersed in 1 mL of a MES buffer (100 mM, pH 5.0), and 15 μg of anti-TSH antibody (manufactured by Funakoshi Co., Ltd.) was added. After incubation at room temperature for 12 hours, the beads were separated using magnetism to remove a supernatant. The beads were washed 5 times with a TBS-T (0.05 mass % Tween 20) buffer to obtain antibody-binding beads. The amount of bound antibody was measured by BCA Assay.

The results are shown in Table 1.

TABLE 1

| | GROUP | | | | | | | | |
|---|---|---|---|---|---|---|---|---|---|
| | LINKING | | | | | | | | |
| | GROUP | | | | | REACTIVE | | | AMOUNT |
| | COMPRISING | | | | | FUNCTIONAL | | EFFECT OF | OF BOUND |
| | REACTIVE | | | | | GROUP | | SUPPRESSING | ANTIBODY |
| | FUNCTIONAL | | | | POLYMER DENSITY | AMOUNT | COLOR | NONSPECIFIC | (μg/mg |
| | GROUP | Mn | Mw | Mw/Mn | (polymers/nm²) | (μmol/g) | CHANGE | ADSORPTION | beads) |
| EXAMPLE 1 | IMINO | 26,800 | 29,480 | 1.1 | 1.0 | 3 | NONE | A | 2 |
| | GROUP | | | | | | | | |
| EXAMPLE 2 | IMINO | 10,200 | 11,220 | 1.1 | 1.0 | 3 | NONE | A | 2 |
| | GROUP | | | | | | | | |
| EXAMPLE 3 | IMINO | 15,000 | 18,000 | 1.2 | 1.0 | 3 | NONE | A | 2 |
| | GROUP | | | | | | | | |
| EXAMPLE 4 | IMINO | 26,000 | 28,600 | 1.1 | 0.3 | 1 | NONE | B | 1 |
| | GROUP | | | | | | | | |
| COMPARATIVE | THIO GROUP | 26,800 | 29,480 | 1.1 | 1.0 | — | YES | — | — |
| EXAMPLE 1 | | | | | | | | | |
| COMPARATIVE | IMINO | 11,000 | 12,100 | 1.1 | 0.08 | 0.5 | NONE | C | 0.5 |
| EXAMPLE 2 | GROUP | | | | | | | | |
| COMPARATIVE | — | — | — | — | — | 10 | — | C | 5 |
| EXAMPLE 3 | | | | | | | | | |

As shown in Table 1, color changes were observed in the magnetic beads in Comparative Example 1 as compared to prior to converting the end structure. The color was changed from blackish color to yellow brownish color, and it is supposed that this change in color was caused by the formation of γ-Fe₂O₃ by reduction of Fe₃O₄ in a magnetic substance by mercaptopropionic acid.

In the magnetic beads in Comparative Example 2, nonspecific adsorption easily occurred and further a ligand did not sufficiently bind thereto. In addition, in the magnetic beads in Comparative Example 3, nonspecific adsorption easily occurred.

In contrast, in the magnetic beads in Examples 1 to 4, degeneration accompanied by color changes was not brought about, and further nonspecific adsorption was suppressed, and the amount of bound ligand was great in spite of the beads having a reactive functional group introduced in the end.

What is claimed is:

1. A magnetic bead, formed by binding a chain polymer at least to the surface,
wherein the chain polymer is a chain polymer which has a hydrophilic repeating unit, and has a group comprising a reactive functional group at the end of the side, to which the magnetic bead does not bind, through an imino group or an N-substituted imino group, and
a density of the chain polymer occupying the surface of the magnetic beads is 0.1 polymers/nm² or more.

2. The magnetic bead according to claim 1, wherein the hydrophilic repeating unit is a repeating unit having a hydrophilic group selected from the group consisting of a hydroxy group, an alkoxy group, a polyoxyalkylene group, a group having a zwitterionic structure, a sulfonyl group, a sulfinyl group and a phosphate group.

3. The magnetic bead according to claim 1, wherein the reactive functional group is a reactive functional group selected from the group consisting of a carboxy group, a tosyl group, an amino group, an epoxy group, an acyl group and an azide group.

4. The magnetic beads according to claim 1, wherein a content of the reactive functional group is from 0.7 to 50 μmol per g of solid content in the magnetic bead.

5. The magnetic bead according to claim 1, wherein the density of the chain polymer occupying the surface of the magnetic bead is from 0.1 to 2 polymers/nm².

6. The magnetic bead according to claim 1, wherein the chain polymer is a hydrophilic polymer.

7. The magnetic bead according to claim 1, wherein the chain polymer is a homopolymer of the hydrophilic repeating unit.

8. The magnetic bead according to claim 1, wherein the molecular weight distribution of the chain polymer is from 1.0 to 2.5.

9. The magnetic bead according to claim 1, wherein the number average molecular weight of the chain polymer is from 1,000 to 100,000.

10. A ligand-binding bead, formed by binding a ligand to the magnetic bead according to claim 1.

11. The ligand-binding bead according to claim 10, wherein the ligand is an antibody, an antigen, a nucleic acid, a nucleotide, a nucleoside, a protein, a peptide, an amino acid, a polysaccharide, a saccharide, a lipid, a vitamin, medicine, a substrate, a hormone or a neurotransmitter.

12. The magnetic bead or the ligand-binding bead according to claim 10 for immunoassay or detecting a nucleic acid.

13. A method for detecting or separating a target substance in a sample, the method being characterized by using the ligand-binding bead according to claim 10.

14. A method for producing the magnetic bead according to claim 1, the method being characterized by comprising
(Step 1) a step of preparing a magnetic bead material having a polymerization initiating group at least on the surface,
(Step 2) a step of polymerizing a hydrophilic monomer from the polymerization initiating group, and
(Step 3) a step of allowing a compound having a primary or secondary amino group to react with the end of a chain polymer formed on the surface of the magnetic bead material in the step 2.

15. The method according to claim 14, further comprising (Step 4) a step of introducing a reactive functional group into the end structure added in the step 3 by an addition reaction, a substitution reaction or a condensation reaction.

16. The production method according to claim 14, wherein the polymerization initiating group is an atom transfer radical polymerization initiating group.
